(19) United States
(12) Patent Application Publication
(10) Pub. No.: US 2026/0198821 A1
(43) Pub. Date: Jul. 16, 2026

(54) Evaluation System and Evaluation Method for Real-Time Fusion of Physiological Data and User Interaction Data to Provide Adaptive Feedback

(71) Applicant: GD-TSENG ENTERPRISE CO., LTD., Taoyuan City (TW)

(72) Inventors: Hsiang-Ting Tseng, Taoyuan City (TW); Oriol Baulenas Muntañola, Granollers (ES)

(21) Appl. No.: 19/024,703

(22) Filed: Jan. 16, 2025

(51) Int. Cl. A61B 5/16; A61B 5/00; A61B 5/024; A61B 5/0533; A61B 5/369; G06V 40/16

(52) U.S. Cl. CPC A61B 5/165; A61B 5/0053; A61B 5/02416; A61B 5/0533; A61B 5/163; A61B 5/369; A61B 5/721; A61B 5/725; G06V 40/174

(57) ABSTRACT

The present disclosure provides an evaluation system and evaluation method for collecting and analyzing multiple streams of physiological data and user interaction data to provide real-time adaptive feedback. A multi-sensor wearable device measures the user's brainwave signals, skin conductance, and optionally collects pupil dilation or eye-tracking data. Simultaneously, an interactive assembly apparatus generates task-related performance data. A computing device fuses these data streams to determine the user's cognitive or emotional state and automatically adjusts the difficulty or instructional content of the task. The system stores the measured data and related feedback in a database, allowing for trend analysis, personalized profiling, and comparisons across multiple tasks or users.

### FIELD OF THE DISCLOSURE

The present disclosure generally relates to interactive systems that monitor user performance and physiological responses, and more specifically, to a system and method for fusing data from multiple sensors—such as electroencephalography (EEG) and galvanic skin response (GSR)—with task-based user interaction data to provide real-time adaptive feedback.

### BACKGROUND OF THE INVENTION

Various interactive systems have been developed to engage users in physical or virtual tasks while tracking their performance. In recent years, wearable devices and smart assembly apparatuses (e.g., programmable blocks or puzzle systems) have become popular for entertainment, educational, or therapeutic purposes. These existing systems often measure basic data, such as the number of errors made while assembling a puzzle, or capture limited physiological parameters (e.g., heart rate).

However, many current solutions fail to fully integrate continuous, multi-channel physiological data (e.g., EEG signals, skin conductance, eye tracking) with real-time performance data, making it impossible to adjust tasks in a highly personalized manner in real-time. Additionally, systems that store such data for subsequent review often lack advanced predictive algorithms capable of detecting trends or precursors to cognitive decline. Therefore, there is a need for a more robust system and method that comprehensively fuses diverse data streams in real-time, providing immediate feedback and long-term analysis for applications in healthcare, training, or other fields.

### SUMMARY OF THE INVENTION

The present disclosure provides an evaluation system and evaluation method for real-time fusion of physiological data and user interaction data, capable of dynamically adjusting adaptive feedback based on the user's cognitive or emotional state. In certain embodiments, a multi-sensor wearable device captures the user's electroencephalography (EEG) signals and galvanic skin response (GSR) data, and may additionally collect eye-tracking or pupil dilation data. Simultaneously, an interactive assembly apparatus, such as a set of smart blocks or a puzzle-based interface, detects the user's actions and captures performance metrics, including assembly accuracy and completion time.

A computing device receives these data streams and processes them through one or more predictive models. By correlating physiological signals with user interaction performance data, the evaluation system can infer the user's cognitive load, emotional state, or stress level in real-time. Based on these inferences, an adaptive feedback mechanism adjusts task parameters—such as increasing or decreasing difficulty, providing in-task hints, or modifying instructional content—to enhance engagement and user outcomes. The evaluation system stores all collected data in a database, allowing for immediate review and longitudinal analysis across multiple tasks. Clinicians, caregivers, or educators can remotely monitor the user's progress and compare current data to previous baselines or peer group benchmarks. In this way, the present disclosure provides an enhanced and robust platform for applications such as training, therapy, or entertainment, which require precise monitoring and real-time adaptive interventions.

Through these and other aspects disclosed herein, the present disclosure addresses limitations of prior systems by offering comprehensive sensor data collection, advanced predictive analytics, real-time feedback loops, and flexible long-term data storage and retrieval.

### BRIEF DESCRIPTION OF THE DRAWINGS

FIG. 1 illustrates a schematic diagram of one embodiment of the evaluation system of the present disclosure.

FIG. 2 illustrates a schematic diagram of one embodiment of the multi-sensor wearable device of the present disclosure.

FIG. 3 illustrates a schematic diagram of another embodiment of the multi-sensor wearable device of the present disclosure.

FIG. 4 illustrates a schematic diagram of one embodiment of the interactive assembly apparatus of the present disclosure.

FIG. 5 illustrates a schematic diagram of one embodiment of the computing device of the present disclosure.

FIG. 6 illustrates a flowchart of one embodiment of the evaluation method of the present disclosure.

### DESCRIPTION OF THE PREFERRED EMBODIMENTS

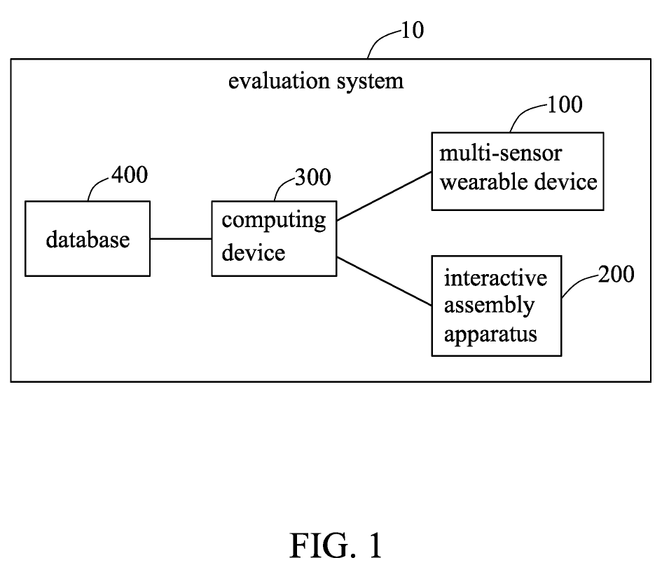

Referring to FIG. 1, FIG. 1 illustrates a schematic diagram of one embodiment of the evaluation system of the present disclosure. In this embodiment, the evaluation system 10 includes a multi-sensor wearable device 100, an interactive assembly apparatus 200, a computing device 300, and a database 400. These components cooperate with each other to provide real-time adaptive feedback based on fused physiological data and user interaction data. This configuration supports a wide range of applications, such as cognitive assessment, therapeutic interventions, and educational training.

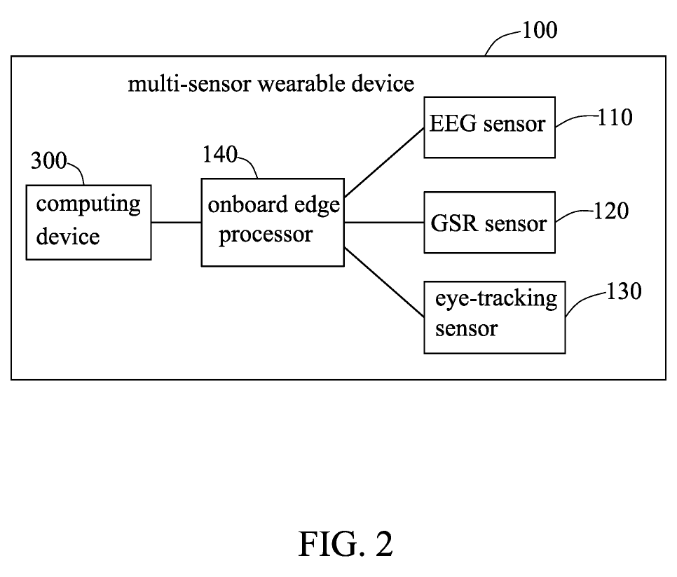

Referring to FIG. 2, FIG. 2 illustrates a schematic diagram of one embodiment of the multi-sensor wearable device of the present disclosure. The multi-sensor wearable device 100 is configured to capture physiological data from user in real time. In one embodiment, the wearable device 100 includes an electroencephalography (EEG) sensor 110 positioned on or near the user’s scalp to measure EEG signals and a galvanic skin response (GSR) sensor 120 that detects changes in the user’s skin conductance indicative of stress or emotional states. In some embodiments, the multi-sensor wearable device 100 may also be equipped with an eye-tracking sensor 130, utilizing an optical sensor or camera to track the user’s gaze direction and pupil changes. In certain embodiments, the wearable device 100 incorporates an onboard edge processor 140 that performs initial filtering of the raw EEG and GSR signals before transmitting them to the computing device 300. This approach enhances data accuracy by reducing noise and stabilizing the signals.

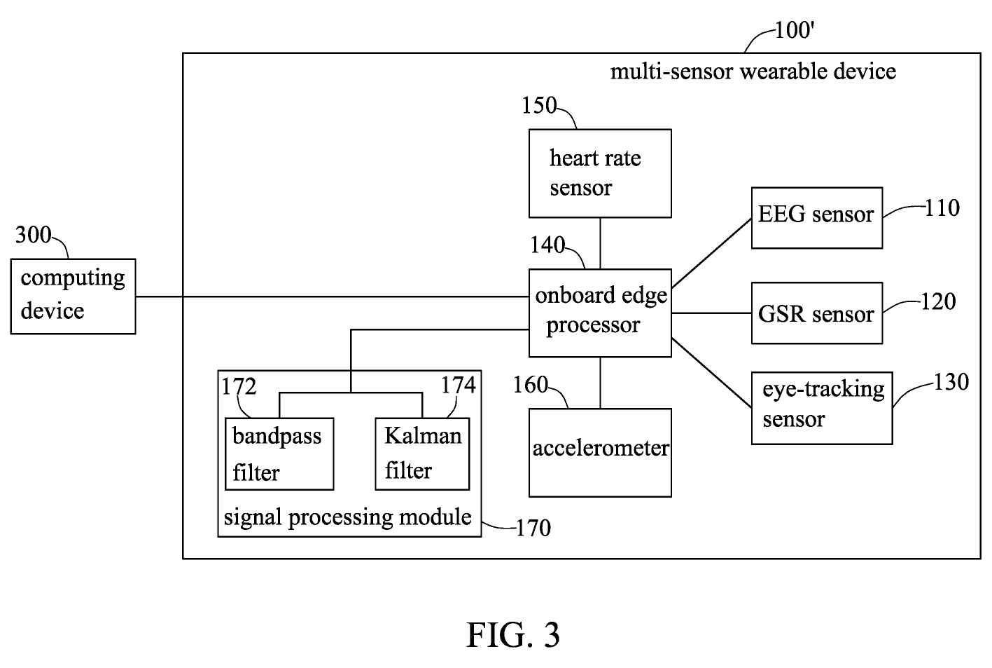

Referring to FIG. 3, FIG. 3 illustrates a schematic diagram of another embodiment of the multi-sensor wearable device of the present disclosure. In certain embodiments of the present disclosure, the multi-sensor wearable device 100’ may incorporate additional biometric sensors beyond the EEG sensor 110 and GSR sensor 120. For example, the wearable device 100 could include a heart rate sensor 150 that non-invasively measures a user’s pulse via photoplethysmography, and an accelerometer 160 that detects the user’s body movements or head motions. In one exemplary configuration, the heart rate sensor 150 is positioned proximate to the user’s temple or wrist, while the accelerometer 160 is embedded within the main housing of the wearable device 100. These additional signals may be captured simultaneously with EEG and GSR data, enabling the evaluation system 10 to form a more holistic profile of the user’s physiological and behavioral states. By analyzing heart rate variability alongside EEG frequency shifts, for instance, the evaluation system 10 can better distinguish between heightened stress reactions and mere physical exertion.

In order to handle the diverse array of data streams, the wearable device 100 or the computing device 300 may implement one or more signal processing modules (in this embodiment, the signal processing module 170 is configured within the computing device 300) that clean, filter, and align these streams in real time. EEG signals, for example, may be passed through a bandpass filter 172, such as 0.5 to 50 Hz, to remove low-frequency drifts and high-frequency noise. In some embodiments, the EEG sensor 110 may be further equipped with artifact detection algorithms 112 that identify and suppress signal disturbances caused by muscle activity, eye blinks, or sudden user movements. The GSR sensor 120 likewise may be subject to baseline correction, ensuring that gradual shifts in skin conductance are reliably differentiated from brief spikes due to transitory stimuli. Meanwhile, any accelerometer signal can be run through a motion artifact reduction algorithm, which flags excessive user movement that could otherwise contaminate the EEG or GSR data.

Once these filtering or artifact rejection processes are complete, the wearable device 100 or computing device 300 can apply data fusion techniques to accurately synchronize the resulting data streams. For example, each signal may be timestamped and placed onto a common timeline, enabling the evaluation system 10 to correlate changes in EEG amplitude or alpha/beta wave ratios with contemporaneous shifts in GSR, heart rate, or user motion. In some embodiments, a Kalman filter 174 or an extended Kalman filter could be employed to merge data from multiple sensors and reduce inherent measurement noise. By integrating these advanced fusion strategies, the evaluation system 10 gains a refined view of the user’s physiological states, which allows the predictive AI module 320 to infer nuanced cognitive or emotional conditions more reliably.

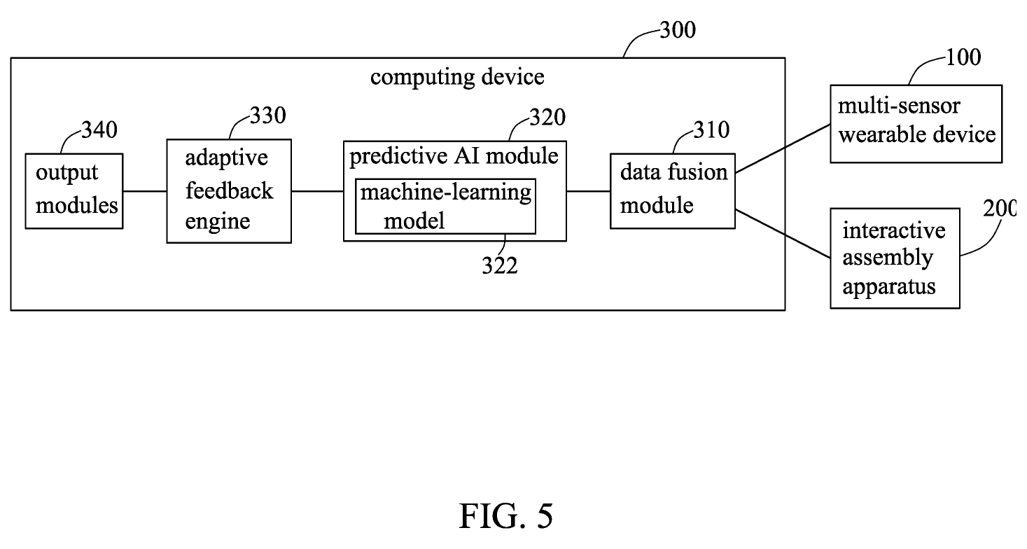

In addition to real-time processing, the data fusion module 310 (as shown in FIG. 5) or an onboard edge processor 140 may maintain calibration routines that adapt to user-specific signal characteristics over repeated interactive tasks. For instance, individuals with naturally higher baseline GSR levels or faster resting heart rates might benefit from personalized filtering thresholds or artifact detection algorithms. The evaluation system 10 can store these user-specific calibration parameters in the database 400, ensuring that each subsequent interactive task commences with optimal signal processing settings. In certain embodiments, machine-learning model 322 within the predictive AI module 320 (as shown in FIG. 5) can analyze archived data to improve the accuracy of artifact classification over time, effectively “learning” from previously encountered patterns of noise or drift. This continual refinement further enhances the reliability of real-time feedback generated by the adaptive feedback engine 330.

Through the combination of expanded sensor arrays and robust signal-processing techniques, the present disclosure offers the flexibility to capture an extensive suite of physiological metrics—e.g., EEG signals, GSR readings, heart rate, and motion data—all of which contribute to more comprehensive assessments of a user’s mental and physical status. By minimizing noise and ensuring synchronized data streams, the evaluation system 10 can detect subtle correlations, such as whether a sudden peak in stress correlates with a user’s hand tremor detected by the accelerometer or a concurrent spike in beta-band EEG activity. These correlations inform more precise adaptive interventions, allowing the invention to respond promptly with task adjustments, calming prompts, or other feedback that is tailored to the user’s current cognitive load and physical state.

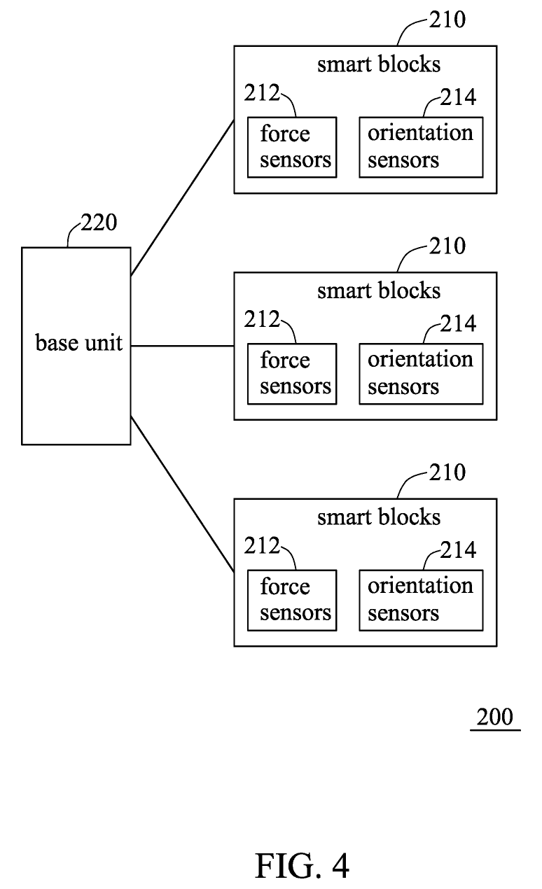

Referring to FIG. 4, FIG. 4 illustrates a schematic diagram of one embodiment of the interactive assembly apparatus of the present disclosure. The interactive assembly apparatus 200 is designed to engage user in physical or virtual tasks. For example, the interactive assembly apparatus 200 may include multiple smart blocks 210, each equipped with force sensors 212 and/or orientation sensors 214 to detect how the smart blocks 210 are placed or rotated. These smart blocks 210 communicate with a base unit 220 via wired or wireless connections, and the base unit 220 collects user performance data such as assembly time, block orientation, accuracy, and error counts. The interactive assembly apparatus 200 can also be implemented as a touchscreen puzzle or a 3D-printed kit that records touch inputs and placement data. Regardless of the form, it generates "assembly data" related to how the user interacts with the task.

Referring to FIG. 5, FIG. 5 illustrates a schematic diagram of one embodiment of the computing device of the present disclosure. The computing device 300, as shown in FIG. 5, receives and processes physiological data from the wearable device 100 and assembly data from the assembly apparatus 200. In one embodiment, it merges the diverse data streams in a data fusion module 310, ensuring that EEG signals, GSR readings, and block assembly data are aligned according to common time references. The predictive AI module 320 analyzes these combined signals to infer the user’s cognitive load or emotional state. This predictive AI module 320 includes a trained machine-learning model 322 that uses features extracted from the EEG, such as the alpha/beta wave ratio, fluctuations in GSR over time, and user interaction patterns, such as speed and accuracy. Based on this analysis, the adaptive feedback engine 330 adjusts difficulty levels, modifies instructions, or provides calming prompts in real time. These feedbacks are delivered through one or more output modules 340, such as a display screen, audio device, or haptic actuator.

In certain embodiments of the present disclosure, the predictive AI module 320 within the computing device 300 goes beyond simple pattern recognition by implementing advanced machine-learning architectures to analyze time-synchronized data from the multi-sensor wearable device 100 and the interactive assembly apparatus 200. This module may employ a combination of neural networks, statistical learning algorithms, or hybrid approaches to extract complex patterns from streams of physiological signals, such as EEG and GSR, along with user-performance metrics like task completion times, error rates, and reaction speeds. One illustrative example involves a deep neural network that ingests a concatenated time window of EEG data—potentially filtered into relevant frequency bands such as alpha, beta, and gamma—and correlates these features with concurrent GSR fluctuations and accelerometer readings (if present). By using multiple convolutional layers, the deep neural network may isolate spatial-temporal features indicative of heightened stress, sustained concentration, or emerging fatigue, which are then integrated into the system’s real-time assessment of the user’s cognitive state.

In an alternative embodiment, the predictive AI module 320 employs recurrent neural networks (RNNs), such as LSTM (Long Short-Term Memory) or GRU (Gated Recurrent Unit) architectures, for capturing dependencies across longer time horizons. These recurrent models track how the user’s physiological and performance indicators evolve during an interactive task, enabling more accurate detection of slow-building stress or progressive learning curves. For instance, if the user’s EEG alpha wave ratio gradually decreases while the GSR remains persistently elevated, the model may infer chronic anxiety rather than a brief startle response. This enhanced temporal sensitivity allows the adaptive feedback engine 330 to proactively adjust puzzle complexity or block-arrangement challenges before the user becomes overwhelmed.

Feature extraction and labeling may be further refined using unsupervised learning techniques, especially when large volumes of unlabeled physiological data are collected in the database 400. Clustering algorithms can segment user states into categories such as “low engagement,” “moderate engagement,” and “high stress,” even without explicit ground truth labels. Once clusters are identified, supervised methods can label or refine them over time based on clinical or educational feedback from caregivers, therapists, or instructors. The system may also incorporate a semi-supervised learning approach in which user-labeled samples (e.g., moments the user presses a “help” button) seed the labeling process, while unlabeled samples are automatically classified by the model as it gains confidence.

In some embodiments, the predictive AI module 320 implements adaptive thresholding algorithms that adjust stress or cognitive-load cutoffs on a user-by-user basis. By analyzing previous interactive tasks, the module identifies each individual’s typical EEG and GSR baselines, tailoring the thresholds accordingly. This personalization helps reduce false positives that could arise from innate physiological differences among users, such as naturally high GSR in warmer climates or a consistently lower resting alpha wave ratio in advanced age groups. Such adaptation may be periodically recalibrated whenever the user or a caregiver observes mismatches between the system’s alerts and the user’s subjective experience.

Model training and updates can occur either locally on the computing device 300 or remotely in a cloud-based environment, depending on computing resources and privacy requirements. In one scenario, the device 300 uploads raw or partially processed sensor data to the database 400 after each interactive task, where a server-side training pipeline incrementally re-trains or fine-tunes the neural networks using fresh data from multiple users. Once improved models are validated, they can be pushed back to each user’s local device. This iterative feedback cycle ensures the AI module continuously evolves and maintains accuracy even as new sensor technologies are introduced or as user populations change.

By leveraging these sophisticated machine-learning strategies, the invention dynamically infers user states with heightened accuracy, detecting subtle cognitive or emotional signals that might elude simpler rule-based systems. The real-time outputs of the predictive AI module 320 inform the adaptive feedback engine 330, enabling more targeted and context-sensitive interventions—whether in the form of calming auditory cues, step-by-step instructions, or intentionally challenging tasks for users who appear under-stimulated. Overall, this fusion of advanced analytics with synchronized multi-sensor data not only strengthens the short-term responsiveness of the system but also provides valuable longitudinal insights for long-term studies of cognitive performance and well-being.

Referring back to FIG. 1, the collected data is stored in the database 400, which may reside locally or on a cloud server. The database 400 holds both raw sensor signals and derived metrics, allowing authorized users to retrieve and analyze the information later. This database 400 facilitates longitudinal studies and comparisons across multiple interactive tasks or multiple users. By maintaining secure, access-controlled files, the system supports healthcare, research, or educational environments where historical performance trends are relevant.

In certain embodiments of the present disclosure, the evaluation system 10 implements advanced privacy and security measures to protect user data during transmission, storage, and processing. In one embodiment, all physiological signals (e.g., EEG, GSR, heart rate) and user interaction data (e.g., assembly actions, performance metrics) transmitted between the multi-sensor wearable device 100 and the computing device 300 are encrypted using the Advanced Encryption Standard (AES) with a 256-bit key, thereby mitigating the risk of unauthorized access or interception. Data stored in the database 400 may also be encrypted at rest via AES-256 encryption, ensuring that sensitive information remains protected even if storage hardware is compromised.

To further safeguard personal identifiable information (PII), the system 10 optionally applies data anonymization techniques before storing or sharing records. In one example, the system employs k-anonymity or similar algorithms to render individual user records non-identifiable, thus reducing the likelihood that an unauthorized party could trace particular physiological data back to any specific user. In addition, role-based access control (RBAC) layers may regulate the permissions of different user roles—such as administrators, clinicians, or researchers—so that only those with appropriate credentials can retrieve or modify sensitive data. A secure user portal further enables individuals to view, correct, or delete their data, while the system maintains logs of all access events or modifications for auditability.

Beyond privacy and security, performance targets in certain embodiments focus on reducing system latency to provide near-real-time adaptive feedback. For instance, the system 10 may guarantee a response time of less than two seconds from the moment EEG or GSR signals are captured and fused with user interaction data to the issuance of an updated task difficulty level. By deploying advanced signal processing and machine learning algorithms, some embodiments maintain a data accuracy rate of at least 95% when classifying user stress states or cognitive load levels. This combination of low-latency responses and robust classification accuracy ensures the evaluation system 10 can deliver timely, reliable feedback to users in diverse operational environments.

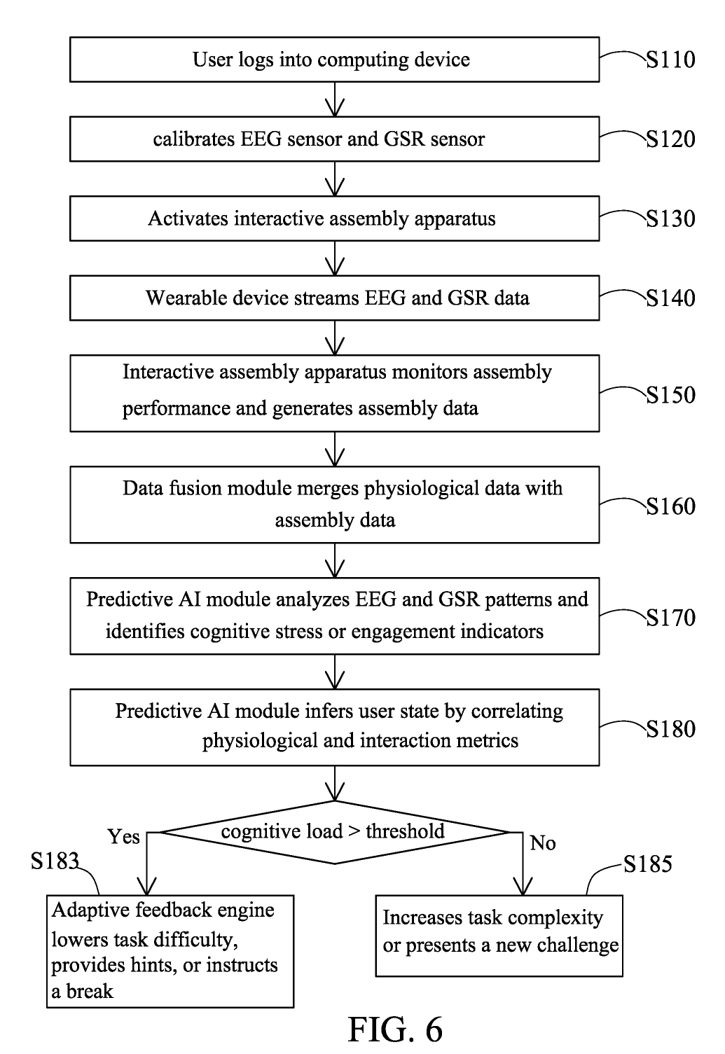

Referring to FIG. 6, FIG. 6 illustrates a flowchart of one embodiment of the evaluation method of the present disclosure. At the start of an interactive task, as shown in step S110, user logs into the computing device 300, which retrieves relevant profile information from the database 400. Then, as shown in step S120, the user wears the wearable device 100, ensuring that the EEG sensor 110 and GSR sensor 120 are properly calibrated. If an eye-tracking sensor 130 is present, it must be aligned to capture meaningful data. Next, as shown in step S130, the interactive assembly apparatus 200 is activated or connected, and any necessary instructions are provided to guide the user. Subsequently, as shown in step S140, the wearable device 100 continuously streams EEG and GSR data, optionally pre-processed to remove noise or artifacts. Meanwhile, as shown in step S150, the interactive assembly apparatus 200 monitors the user’s performance in assembling blocks or solving puzzles, generating "assembly data" that reflects speed, accuracy, time spent, and any notable actions or errors.

In this embodiment, as shown in step S160, the data fusion module 310 of the computing device 300 merges these physiological data streams with the assembly data and timestamps them to maintain proper synchronization. As shown in step S170, the predictive AI module 320 examines patterns in the EEG waveforms and GSR fluctuations, identifying indicators of cognitive stress or engagement. By correlating these physiological signals with user interaction metrics—such as the time taken to place a block or the frequency of errors—as shown in step S180, the predictive AI module 320 infers whether the user is struggling, performing well, or showing signs of fatigue. If the inferred cognitive load exceeds a predetermined threshold, as shown in step S183, the adaptive feedback engine 330 may lower the difficulty of the assembly task, provide hints, or instruct the user to take a short break. Conversely, as shown in step S187, if the evaluation system 10 detects a relatively low stress level and strong performance, it may increase the task’s complexity or present a new challenge.

Throughout the interactive task, these automatic adjustments continue as new data is received from the wearable device 100 and the assembly apparatus 200. This iterative feedback loop provides a highly personalized experience, ensuring that tasks remain neither too simple nor too taxing for the user. The computing device 300 periodically transmits updated sensor data, derived metrics, and user interaction records to the database 400. At the end of the interactive task, the user may receive a summary report detailing average stress levels, cumulative assembly times, error rates, and any relevant trends or anomalies. This summary is also stored in the database 400, allowing the user, caregivers, or medical professionals to retrieve and analyze it later.

In an embodiment for cognitive training of elderly users, the wearable device 100 measures EEG and GSR signals while elderly individuals assemble physical puzzles. The predictive AI module 320 monitors these signals to identify if the user is experiencing unusual stress, confusion, or fatigue. If the detected stress exceeds a threshold, the adaptive feedback engine 330 may provide simpler tasks or add supportive instructions. All data generated by the interactive tasks are stored in the database 400, allowing caregivers to track progress over time and detect early signs of cognitive decline.

In another embodiment targeting children's education, the evaluation system 10 uses smart blocks 210 decorated with different colors and shapes. As children work through color-coded or shape-based puzzles, real-time EEG and GSR readings provide insights into attention span and emotional engagement. When the evaluation system 10 detects a drop in engagement, it triggers more creative mini-challenges or plays supportive audio prompts. This keeps children interested, and their continuous physiological responses confirm the effectiveness of the interventions.

Another embodiment involves a remote therapy scenario where healthcare providers access the database 400 via a secure portal. Therapists can schedule tasks for patients who use the wearable device 100 and assembly apparatus 200 at home. During the task, if the evaluation system 10 identifies increased anxiety or cognitive overload, it can immediately adjust the task. Therapists can review quantitative data—such as EEG waveforms and assembly completion rates—and qualitative measures, including recorded video of the user’s facial expressions or body language, to tailor subsequent therapy tasks.

Although specific embodiments have been described, those skilled in the art will recognize that variations and modifications can be made without departing from the overall spirit of the invention. For example, the wearable device 100 may integrate additional sensors, such as heart rate monitors or accelerometers, while the interactive apparatus 200 might be implemented in virtual reality or augmented reality environments instead of physical blocks. The computing device 300 can also employ various machine-learning methods, ranging from traditional statistical methods to deep neural networks, depending on system requirements and available computing resources.

Through the combination of multi-sensor data collection, AI-driven analysis, and adaptive feedback mechanisms, this evaluation system provides a powerful means to dynamically adjust tasks based on the user’s cognitive load or stress levels. By integrating real-time assembly data with simultaneous physiological monitoring, the invention not only provides immediate support to the user but also offers a rich data record for long-term analysis in training, therapeutic, and research environments. This integrated, iterative approach distinguishes the invention from traditional systems that rely on limited data streams or delayed offline processing.

1. An evaluation system for collecting and analyzing user interaction and physiological data to provide adaptive feedback, comprising:
a multi-sensor wearable device, comprising:
an electroencephalography sensor configured to measure the user's brainwave signals; and
a galvanic skin response sensor configured to measure skin conductance indicative of the user's stress level;
an interactive assembly apparatus configured to detect the user's assembly actions and generate corresponding assembly data;
a computing device in communication with the multi-sensor wearable device and the interactive assembly apparatus, the computing device configured to:
receive and fuse the brainwave signals, the skin conductance, and the assembly data;
analyze the fused data in real time to generate a cognitive state estimate of the user; and
transmit adaptive feedback to the user via at least one output module based on the cognitive state estimate;
wherein the computing device is further configured to store the brainwave signals, the skin conductance, the assembly data, and the cognitive state estimate in a database for subsequent retrieval and review.

2. The evaluation system of claim 1, wherein the multi-sensor wearable device further comprises an eye-tracking sensor configured to detect gaze direction or pupil dilation, and wherein the computing device fuses the gaze direction or pupil dilation with the brainwave signals, the skin conductance, and the assembly data.

3. The evaluation system of claim 1, wherein the interactive assembly apparatus comprises a plurality of smart blocks, each smart block including at least one force sensor configured to detect force or pressure applied by the user during assembly of the smart blocks.

4. The evaluation system of claim 1, wherein the adaptive feedback comprises automatically adjusting the difficulty level of the interactive assembly task by modifying instructions displayed on a screen.

5. The evaluation system of claim 1, further comprising a camera configured to capture video of the user, wherein the computing device is configured to analyze the user's facial expressions and further refine the cognitive state estimate based on the facial expressions.

6. The evaluation system of claim 1, wherein the computing device comprises a predictive model trained to identify the potential onset of cognitive decline by analyzing historical trends in the fused data across multiple user tasks.

7. The evaluation system of claim 1, wherein the multi-sensor wearable device further comprises a heart rate sensor configured to measure the user’s pulse via photoplethysmography, and wherein the computing device is configured to fuse heart rate data with the brainwave signals, the skin conductance, and the assembly data to generate a refined cognitive state estimate.

8. The evaluation system of claim 1, wherein the multi-sensor wearable device further comprises an accelerometer configured to detect motion artifacts, and wherein the computing device is configured to reduce noise in the brainwave signals or the skin conductance based on motion data from the accelerometer.

9. The evaluation system of claim 1, wherein the computing device is further configured to implement one or more filtering or artifact detection algorithms that apply bandpass filtering to the brainwave signals and baseline correction to the skin conductance to enhance the accuracy of the cognitive state estimate.

10. The evaluation system of claim 1, wherein the predictive model is a recurrent neural network trained to detect user stress or cognitive overload by analyzing historical dependencies in the fused data, the predictive model comprising at least one of a Long Short-Term Memory or Gated Recurrent Unit architecture.

11. The evaluation system of claim 1, wherein the computing device is further configured to establish a user-specific calibration routine that adjusts threshold levels for stress detection based on multiple prior sessions, the user-specific calibration routine being stored in the database and updated over time.

12. An evaluation method for adaptively adjusting a user-interactive task based on real-time physiological signals, comprising:
detecting a physiological signal of a user via at least one sensor, the physiological signal including electroencephalography data and galvanic skin response data;
receiving assembly data representing the user's actions in assembling one or more components via an interactive assembly apparatus;
analyzing the electroencephalography data, the galvanic skin response data, and the assembly data via a predictive model implemented on a computing device to generate a cognitive load metric;
comparing the cognitive load metric to a predetermined threshold;
based on the comparison, modifying the difficulty level of the user-interactive task to obtain an updated user-interactive task; and
storing at least the electroencephalography data, the galvanic skin response data, the assembly data, and the cognitive load metric in a database for subsequent retrieval and review.

13. The evaluation method of claim 12, further comprising capturing a video feed of the user during execution of the user-interactive task, and analyzing the video feed to detect one or more facial expressions associated with emotional states.

14. The evaluation method of claim 12, wherein modifying the difficulty level comprises providing a simplified assembly instruction or an on-screen hint in response to an elevated cognitive load metric.

15. The evaluation method of claim 12, further comprising presenting the updated user-interactive task to the user via a display device, wherein the updated user-interactive task includes at least one prompt derived from prior user performance.

16. The evaluation method of claim 12, further comprising sharing the stored electroencephalography data, galvanic skin response data, and assembly data with a remote server for aggregation and cross-user analysis.

17. The evaluation method of claim 12, further comprising measuring at least one additional physiological signal selected from the group consisting of heart rate and user body motion, and fusing the at least one additional physiological signal with the electroencephalography data and the galvanic skin response data to improve the accuracy of the cognitive load metric.

18. The evaluation method of claim 12, further comprising filtering the electroencephalography data with a bandpass filter and performing baseline correction on the galvanic skin response data to reduce artifacts or noise prior to analyzing the electroencephalography data, the galvanic skin response data, and the assembly data via the predictive model.

19. The evaluation method of claim 12, further comprising adapting a stress threshold for the user-interactive task based on user-specific historical data stored in the database, wherein the stress threshold is automatically recalibrated if mismatches are detected between the user’s subjective experience and the predictive model’s inferences.

20. The evaluation method of claim 12, further comprising training or updating the predictive model in a cloud-based environment using aggregated data from multiple users, and thereafter deploying an updated model to the computing device for real-time inference during subsequent user-interactive tasks.
